(19) United States
(12) Patent Application Publication
(10) Pub. No.: US 2013/0064896 A1
(43) Pub. Date: Mar. 14, 2013

(54) Gastroretentive Solid Oral Dosage Forms with Swellable Hydrophilic Polymer

(71) Applicants: Laman Lynn Alani, Hillsborough, CA (US); Jim H. Kou, San Jose, CA (US); Shook-Fong Chin, Hayward, CA (US); Guangbin Ding, Fremont, CA (US); Natasha G. Masand, Fremont, CA (US)

(72) Inventors: Laman Lynn Alani, Hillsborough, CA (US); Jim H. Kou, San Jose, CA (US); Shook-Fong Chin, Hayward, CA (US); Guangbin Ding, Fremont, CA (US); Natasha G. Masand, Fremont, CA (US)

(21) Appl. No.: 13/519,093

(22) PCT Filed: Dec. 28, 2010
(86) PCT No.: PCT/US10/62262

§ 371 Date: Oct. 11, 2012

Related U.S. Application Data
(60) Provisional application No. 61/290,819, filed on Dec. 29, 2009.

(51) Int. Cl. A61K 9/14; A61K 31/198; A61J 3/02; A61K 31/197

(52) U.S. Cl. 424/497; 424/400; 514/567; 424/490; 264/15

(57) ABSTRACT

The disclosure provides multiparticulate systems that give release of active agents with a narrow window of absorption such that there is bioavailability to a patient. The disclosure provides a composition comprising microparticulates comprising a swellable hydrophilic polymer and an active agent, wherein the swellable hydrophilic polymer is substantially non-crosslinked intramolecularly, and the size of the microparticulates is about 500 μm or less.

### BACKGROUND

Many active agents that are orally administered are absorbed in the upper part of the gastrointestinal tract, which constitutes the “window of absorption.” The duration of passage of the active agent through this window is limited in time. Consequently, the absorption time is itself may be limited. Formulations of active agents that are designed to prolong the exposure of the formulation, and therefore the active, in the upper GI tract may provide a longer period of absorption of the active.

### SUMMARY

This disclosure provides multiparticulate systems for oral delivery of an activeagent, which multiparticulate systems can facilitate prolonged release of active agent over the narrow window of absorption of the upper GI tract.

The multiparticulate systems using a swellable hydrophilic polymer can provide increased residence time of an active agent in the upper gastrointestinal (GI) tract as compared to an active agent without such a multiparticulate system. The multiparticulate systems containing a hydrophilic polymer can swell and form a gel. The swellable hydrophilic polymer can also contain air pockets which can be formed within the swollen granules. Thus, the particulates tend to float in the fluid in the gastric environment and escape the gastric emptying wave. Also, these multiparticulate systems can prolong the GI transit time of an active agent with small particle sizes in which the particulates become trapped in the folds of the stomach and between the villae of the small intestine. The active agent release from multiparticulate systems using a swellable hydrophilic polymer takes place as a combination of diffusion and erosion of the particulates.

The disclosure also provides a composition comprising microparticulates comprising a swellable hydrophilic polymer and an active agent, wherein the swellable hydrophilic polymer is substantially non-crosslinked intramolecularly; and the size of the microparticulates is about 500 μm or less. In certain embodiments, the size of the microparticulates is about 300 μm or less. In some embodiments, the composition does not include a gas-generating agent.

The release profile of the composition can be assessed by the paddle method with simulated gastric fluid (SGF). In certain embodiments, the composition releases about 40% to about 60% of the drug within about 4 hours. In certain embodiments, the composition releases about 70% to about 90% of the drug within about 8 hours. In certain embodiments, the composition releases about 80% to about 95% of the drug within about 12 hours.

### BRIEF DESCRIPTION OF THE FIGURES

FIG. 1 shows dissolution profiles of a multiparticulate system comprising baclofen and HPMC that was obtained through the mixing/micronization procedure with different amounts of swellable hydrophilic polymer.

FIG. 2 shows dissolution profiles of a multiparticulate system comprising baclofen multiparticulate system using a swellable hydrophilic polymer that was obtained through the coated procedure.

FIG. 3 shows dissolution profiles of a multiparticulate system comprising baclofen and a swellable hydrophilic polymer that was obtained through the mixing/micronization procedure or coated procedure.

FIG. 4 shows dissolution profiles of a multiparticulate system comprising baclofen and a swellable hydrophilic polymer in different dissolution media.

FIG. 5 shows dissolution profiles of a multiparticulate system comprising baclofen and a swellable hydrophilic polymer as tested by the basket method and paddle method.

FIG. 6 shows dissolution profiles of a multiparticulate system comprising levodopa and HPMC that was obtained through the mixing/micronization procedure.

FIG. 7 shows dissolution profiles of a multiparticulate system comprising levodopa and a swellable hydrophilic polymer as tested by the basket method and paddle method.

### DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a,” “an,” and “the” include plural referents unless the context clearly dictates otherwise. It is further noted that the claims may be drafted to exclude any optional element.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The multiparticulate systems using a swellable hydrophilic polymer can provide for increased residence time of active agent in the upper gastrointestinal (GI) tract as compared to an active agent without a multiparticulate system. The multiparticulate systems containing a hydrophilic polymer can swell and form a gel. The swellable hydrophilic polymer can also contain air pockets which can be formed within the swollen granules. Thus, the particulates tend to float in the fluid in the gastric environment and escape the gastric emptying wave. Also, these multiparticulate systems can prolong the GI transit time of an active agent with small particle sizes in which the particulates become trapped in the folds of the stomach and between the villae of the small intestine. The active agent release from multiparticulate systems using a swellable hydrophilic polymer takes place as a combination of diffusion and erosion of the particulates.

The term “microparticulate” refers to discrete particles, which may be solid or semisolid at room temperature, and which are generally of a size of 500 μm or less or 300 μm or less and usually at least 10 μm.

The term “multiparticulate system” refers to dosage forms comprising a multiplicity of discrete units, each exhibiting some desired characteristics. In these systems, the dosage is divided into a plurality of units.

### Multiparticulate Systems Using Swellable Hydrophilic Polymers

The multiparticulate systems using a swellable hydrophilic polymer can provide for increased residence time of active agent in the upper gastrointestinal (GI) tract as compared to an active agent without a multiparticulate system. The multiparticulate systems containing a hydrophilic polymer can swell and form a gel. The swellable hydrophilic polymer can also contain air pockets which can be formed within the swollen granules. Also, the GI transit time of these multiparticulate systems can be prolonged when the particle sizes are small enough to allow the particulates to become trapped in the folds of the stomach and between the villae of the small intestine. The active agent's release from multiparticulate systems using a swellable hydrophilic polymer takes place as a combination of diffusion and erosion of the particulates.

The release profile of the composition can be assessed by the paddle method with simulated gastric fluid (SGF). In certain embodiments, the composition releases about 40% to about 60% of the drug within about 4 hours. In certain embodiments, the composition releases about 70% to about 90% of the drug within about 8 hours. In certain embodiments, the composition releases about 80% to about 95% of the drug within about 12 hours.

As noted herein, in certain embodiments of the present disclosure, the multiparticulate systems do not include a gas-generating agent. A “gas-generating agent” refers to a substance known to produce carbon dioxide or sulfur dioxide upon contact with gastric fluid. Examples of gas-generating agents that produce carbon dioxide include sodium or potassium hydrogen carbonate, calcium carbonate, sodium glycine carbonate. Examples of gas-generating agents that produce sulfur dioxide include sulfur sulfite, sodium bisulfite, and sodium metabisulfite.

Examples of swellable hydrophilic polymers and active agents are described below.

### Swellable Hydrophilic Polymers

The embodiments provide a composition comprising microparticulates comprising a swellable hydrophilic polymer and an active agent, wherein the swellable hydrophilic polymer is substantially non-crosslinked intramolecularly; and the size of the microparticulates is about 500 μm or less. In certain embodiments, the size of the microparticulates is about 300 μm or less.

The swellable hydrophilic polymer is non-toxic and can swell in a dimensionally unrestricted manner upon imbibition of water, and can provide for sustained-release of an incorporated active agent.

Examples of suitable polymers include, for example, cellulose polymers and their derivatives (such as for example, hydroxyethylcellulose, hydroxypropylcellulose, carboxymethylcellulose, and microcrystalline cellulose), polysaccharides and their derivatives, polyalkylene oxides, polyethylene glycols, chitosan, poly(vinyl alcohol), xanthan gum, maleic anhydride copolymers, poly(vinyl pyrrolidone), starch and starch-based polymers, poly(2-ethyl-2-oxazoline), poly(ethyleneimine), polyurethane hydrogels, gums, alginates, lectins, carbopol, and combinations comprising one or more of the foregoing polymers.

In certain embodiments, the swellable hydrophilic polymer is cellulose and derivatives thereof. All alkyl-substituted cellulose derivatives in which the alkyl groups have 1 to 3 carbon atoms, prderably 2 carbon atoms, and having suitable properties as noted are contemplated. Cellulose is used herein to mean a linear polymer of anhydroglucose. In general, suitable alkyl-substituted celluloses have a mean viscosity from about 1,000 to 4,000 centipoise (1% aqueous solution at 20° C.); other suitable alkyl-substituted celluloses may fall in a viscosity range from about 100 to 6,500 centipoise (2% aqueous solution at 20° C.).

Examples of swellable hydrophilic polymers that are cellulose and derivatives thereof include, but not limited to, cellulose (such as microcrystalline cellulose), hydroxymethylcellulose, hydroxyethylcellulose (HEC), hydroxypropylmethylcellulose (HPMC), hydroxypropycellulose (HPC), methylcellulose (MC or METHOCEL), ethylcellulose (EC), hydroxyethylmethylcellulose (HEMC), ethylhydroxy-ethylcellulose (EHEC), and carboxymethylcellulose.

Suitable polyalkylene oxides are those having the properties described above for alkyl-substituted cellulose polymers. An example of a polyalkylene oxide is poly(ethylene oxide), which term is used herein to denote a linear polymer of unsubstituted ethylene oxide. Poly(ethylene oxide) polymers having molecular weights of about 4,000,000 and higher are particularly suitable. More preferred are those with molecular weights of about 4,500,000 to about 10,000,000, and even more preferred are polymers with molecular weights of about 5,000,000 to about 8,000,000. Preferred poly(ethylene oxide)s are those with a weight-average molecular weight of about 1×10⁵ to about 1×10⁷, such as within the range of about 9×10⁵ to about 8×10⁶. Poly(ethylene oxide)s are often characterized by their viscosity in solution. A certain viscosity is about 50 to about 2,000,000 centipoise for a 2% aqueous solution at 20° C. Two examples of poly(ethylene oxide)s are POLYOX™ NF, grade WSR Coagulant, molecular weight 5 million, and grade WSR 303, molecular weight 7 million, both available from Dow.

Polysaccharide gums, both natural and modified (semi-synthetic) can be used. Examples are dextran, xanthan gum, gellan gum, welan gum and rhamsan gum.

### Optional Controlled Release Coating

The multiparticulate system can optionally include a controlled release coating. Examples of a suitable controlled release polymers are EUDRAGIT® polymers which are poly(meth)acrylates. Certain EUDRAGIT® polymers include EUDRAGIT® NE grade, EUDRAGIT® NM grade, EUDRAGIT® RL grade, and EUDRAGIT® RS grade.

Certain other suitable controlled release polymers include hydrophobic controlled release polymer coatings, such as ethyl cellulose. Certain other suitable controlled release polymers include enteric coatings, such as EUDRAGIT® L 100 and EUDRAGIT® L 100-55. Certain other suitable controlled release polymers include neutral controlled release polymer coatings, such as EUDRAGIT® NE 30 D and KOLLIDON®.

### Particle Sizes for Multiparticulate Systems Using Swellable Hydrophilic Polymers

The multiparticulate system using a swellable hydrophilic polymer employs fine particles with particle sizes of about 500 μm or less. In certain embodiments, the size of the microparticulates is about 300 μm or less. The particulate size is taken when the multiparticulate system comprises a swellable hydrophilic polymer and an active agent.

In certain embodiments, the particle size ranges disclosed herein indicate the particle size range of 90% of the particles in the composition comprising the drug-resin complexes.

In the discussion below, if not specified, the lower end of the range is at least 10 μm and can be about 50 μm.

In certain embodiments, the particle size is about 480 μm or less. In certain embodiments, the particle size is about 460 μm or less. In certain embodiments, the particle size is about 450 μm or less. In certain embodiments, the particle size is about 440 μm or less. In certain embodiments, the particle size is about 420 μm or less. In certain embodiments, the particle size is about 400 μm or less.

In certain embodiments, the particle size is about 380 μm or less. In certain embodiments, the particle size is about 360 μm or less. In certain embodiments, the particle size is about 350 μm or less. In certain embodiments, the particle size is about 340 μm or less. In certain embodiments, the particle size is about 320 μm or less. In certain embodiments, the particle size is about 300 μm or less.

In certain embodiments, the particle size is about 280 μm or less. In certain embodiments, the particle size is about 260 μm or less. In certain embodiments, the particle size is about 250 μm or less. In certain embodiments, the particle size is about 240 μm or less. In certain embodiments, the particle size is about 220 μm or less. In certain embodiments, the particle size is about 200 μm or less.

In certain embodiments, the particle size is about 180 μm or less. In certain embodiments, the particle size is 160 μm or less. In certain embodiments, the particle size is about 150 μm or less. In certain embodiments, the particle size is about 140 μm or less. In certain embodiments, the particle size is about 120 μm or less.

In certain embodiments, the particle size range is from about 100 μm to about 500 μm. In certain embodiments, the particle size range is from about 100 μm to about 475 μm. In certain embodiments, the particle size range is from about 100 μm to about 450 μm. In certain embodiments, the particle size range is from about 100 μm to about 425 μm.

In certain embodiments, the particle size range is from about 100 μm to about 400 μm. In certain embodiments, the particle size range is from about 100 μm to about 375 μm. In certain embodiments, the particle size range is from about 100 μm to about 350 μm. In certain embodiments, the particle size range is from about 100 μm to about 325 μm.

In certain embodiments, the particle size range is from about 100 μm to about 300 μm. In certain embodiments, the particle size range is from about 100 μm to about 275 μm. In certain embodiments, the particle size range is from about 100 μm to about 250 μm. In certain embodiments, the particle size range is from about 100 μm to about 225 μm. In certain embodiments, the particle size range is from about 100 μm to about 200 μm.

In certain embodiments, the particle size range is from about 475 μm to about 500 μm. In certain embodiments, the particle size range is from about 450 μm to about 500 μm. In certain embodiments, the particle size range is from about 425 μm to about 500 μm. In certain embodiments, the particle size range is from about 400 μm to about 500 μm. In certain embodiments, the particle size range is from about 375 μm to about 500 μm. In certain embodiments, the particle size range is from about 350 μm to about 500 μm. In certain embodiments, the particle size range is from about 325 μm to about 500 μm. In certain embodiments, the particle size range is from about 300 μm to about 500 μm.

In certain embodiments, the particle size range is from about 375 μm to about 400 μm. In certain embodiments, the particle size range is from about 350 μm to about 400 μm. In certain embodiments, the particle size range is from about 325 μm to about 400 μm. In certain embodiments, the particle size range is from about 300 μm to about 400 μm. In certain embodiments, the particle size range is from about 275 μm to about 400 μm. In certain embodiments, the particle size range is from about 250 μm to about 400 μm. In certain embodiments, the particle size range is from about 225 μm to about 400 μm. In certain embodiments, the particle size range is from about 200 μm to about 400 μm.

In certain embodiments, the particle size range is from about 275 μm to about 300 μm. In certain embodiments, the particle size range is from about 250 μm to about 300 μm. In certain embodiments, the particle size range is from about 225 μm to about 300 μm. In certain embodiments, the particle size range is from about 200 μm to about 300 μm. In certain embodiments, the particle size range is from about 175 μm to about 300 μm. In certain embodiments, the particle size range is from about 150 μm to about 300 μm. In certain embodiments, the particle size range is from about 125 μm to about 300 μm.

### Examples of Combinations

It will be appreciated from above that the disclosure provides a multiparticulate system comprising a swellable hydrophilic polymer and an active agent. Examples of multiparticulate systems containing a swellable hydrophilic polymer and an active agent are described below.

In certain embodiments, the multiparticulate system comprises an active agent and HPMC.

In certain embodiments, the multiparticulate system comprises an active agent and microcrystalline cellulose.

In certain embodiments, the multiparticulate system comprises an active agent and ethyl cellulose.

In certain embodiments, the multiparticulate system comprises an active agent and carbopol polymer.

In certain embodiments, the multiparticulate system comprises an active agent and carboxymethylcellulose.

### Active Agents

The terms “active agent” or “active pharmaceutical agent” refers either to a medicinal substance intended, after administration, to bring about a preventive or therapeutic response, or to a combination of two or more substances of this type.

In certain embodiments, the active agent has an absorption that occurs mainly in the upper parts of the gastrointestinal tract. These active agents have a limited window of absorption.

According to the biopharmaceutical classification of drugs in terms of their solubility and intestinal permeability by the FDA, drugs are categorized into four classes. Class I compounds are defined as those with high solubility and high permeability, and are predicted to be well absorbed when given orally. The other classes, Classes II-IV, suffer from low solubility, low permeability, or both and display variable absorption in different regions of the GI tract and as a consequence, their oral bioavailabilities can be affected by the limited absorption window.

In certain embodiments, the active agent is a compound from Classes II-IV, according to the biopharmaceutical classification of drugs in terms of their solubility and intestinal permeability by the FDA. In certain embodiments, the active agent is a compound from Class I, according to the biopharmaceutical classification of drugs in terms of their solubility and intestinal permeability by the FDA.

The absorption of active agents can be limited by reduced solubility or lack of solubility of an active agent. In certain embodiments, an active agent has reduced solubility or lack of solubility in gastric fluid or water.

The absorption of active agents can also be limited by the active transport mechanism in the upper GI tract for absorption. Certain active agents may use active transport mechanism from the upper GI tract, but are poorly absorbed in the large intestine (or colon). As a consequence, the oral bioavailability can be affected by the limited absorptive site. In certain embodiments, an active agent is a compound that uses active transport mechanism in the upper GI tract.

The active agent can be present as different physical forms. Examples of different physical forms of the active agent include, but are not limited to, pharmaceutically acceptable salts, solvates, co-crystals, polymorphs, hydrates, solvates of a salt, co-crystals of a salt, amorphous, and the free form of the active agent.

In certain embodiments, the active agent is baclofen. When referring to baclofen, the active agent may be in the salt form or the base form (e.g., free base). Further, baclofen may be in the salt form and one well-known commercially available salt for baclofen is its hydrochloride salt. Some other examples of potentially pharmaceutically acceptable salts include basic salt forms, such as its sodium salt and tetrabutylammonium salt.

In certain embodiments, the active agent is levodopa or a salt thereof. When referring to levodopa, the active agent may be in the salt form or the free form. Levodopa may be commercially available in the free form.

Certain active agents that have a limited window of absorption include, but are not limited to, acyclovir, bisphosphonates, captopril, furosemide, metformin, gabapentin, ciprofloxacin, cyclosporine, allopurinol, chlordiazepoxide, cinnarizine, and misoprostol.

Preparation of Microparticulates with Swellable Hydrophilic Polymers

The microparticulates with a swellable hydrophilic polymer can be prepared by methods discussed below, including mixing method, coating method, and wet granulation method.

Mixing Method with Optional Micronization

For certain mixing method, a solid swellable hydrophilic polymer and solid active agent are mixed together. Additional additives can be added to the mixture. The mixture can be encapsulated.

Thus, the disclosure provides a method of preparing a composition comprising microparticulates comprising a swellable hydrophilic polymer and an active agent, wherein the swellable hydrophilic polymer is substantially non-crosslinked intramolecularly; and the size of the microparticulates is about 500 μm or less, the method comprising mixing solid swellable hydrophilic polymer and solid active agent.

For certain embodiments, an active agent and/or a swellable hydrophilic polymer can be micronized or size-reduced before mixing the components together. In certain embodiments, an active agent is micronized or size-reduced before mixing the components together. In certain embodiments, a swellable hydrophilic polymer is micronized or size-reduced before mixing the components together. For example, an active agent and/or a swellable hydrophilic polymer can be milled. Then, the active agent and the swellable hydrophilic polymer are mixed together. Additional additives can be added to the mixture.

The mixture of active agent and swellable hydrophilic polymer can be granulated to help blend the components. Granulation can be performed, for example, with a high shear granulator, twin shell blender or double-cone blender, or a simple planetary mixer. The granulated mixture can be screened through a suitably sized mesh screen. A Fitzmill or Co-mill or oscillating mill may be used to control granule size. A V-blender or double cone blender may be used for final blending. The mixture can be encapsulated.

### Coating Method

For certain coating methods, a solid swellable hydrophilic polymer is coated with an active agent. The active agent is dissolved in a solution or suspension and coated on the solid swellable hydrophilic polymer. In certain embodiment, the solid swellable hydrophilic polymer is in the form of beads. The coating process can utilize a fluid bed granulation, for example.

For certain other coating methods, nonpareil seeds are coated with an active agent. Nonpareil seeds can be cellulose base or sugar base. In certain embodiments, the nonpareil seeds are solid microcrystalline cellulose beads. The active agent is dissolved or suspended in a solution and coated on the nonpareil seeds. The coating process can utilize a fluid bed granulation, for example. Then, a solid swellable hydrophilic polymer is mixed with the nonpareil seeds coated with active agent.

Thus, the disclosure provides a method of preparing a composition comprising microparticulates comprising a swellable hydrophilic polymer and an active agent, wherein the swellable hydrophilic polymer is substantially non-crosslinked intramolecularly; and the size of the microparticulates is about 500 μm or less, the method comprising dissolving an active agent in a solution or suspension; coating a nonpareil seed with the solution or suspension comprising the active agent; and mixing a solid swellable hydrophilic polymer with the nonpareil seeds coated with active agent.

### Wet Granulation Method

In a certain wet granulation method, an active agent is mixed with a swellable hydrophilic polymer. The mixture of active agent and swellable hydrophilic polymer is wet granulated. In wet granulation, the mixture is mixed with a wetting agent to provide a wet mass and to densify the materials in the mixture. Wet granulation can be performed with a mixer/granulator. A wetting agent is an inert liquid. The wet mass is then extruded. The extrusion can be performed by means of an extrusion granulator. The extrudates are subjected to spheronization to obtain microparticles.

Thus, the disclosure provides a method of preparing a composition comprising microparticulates comprising a swellable hydrophilic polymer and an active agent, wherein the swellable hydrophilic polymer is substantially non-crosslinked intramolecularly; and the size of the microparticulates is about 500 μm or less, the method comprising mixing an active agent with a swellable hydrophilic polymer; wet granulating the mixture of active agent and swellable hydrophilic polymer; extruding the mixture of active agent and swellable hydrophilic polymer; and subjecting the mixture of active agent and swellable hydrophilic polymer to spheronization to obtain microparticles.

In another wet granulation method, an active agent is mixed with an inert polymer to be wet granulated. Certain inert polymers include microcrystalline cellulose and sugars, such as lactose. The wet mass is then extruded. The extrusion can be performed by means of an extrusion granulator. The extrudates are subjected to spheronization to obtain microparticles. Then the microparticles of active agent and inert polymer are blended with swellable hydrophilic polymer.

Thus, the disclosure provides a method of preparing a composition comprising microparticulates comprising a swellable hydrophilic polymer and an active agent, wherein the swellable hydrophilic polymer is substantially non-crosslinked intramolecularly; and the size of the microparticulates is about 500 μm or less, the method comprising mixing an active agent with an inert polymer; wet granulating the mixture of active agent and inert polymer; extruding the mixture of active agent and inert polymer; subjecting the mixture of active agent and inert polymer to spheronization to obtain microparticles; and mixing the microparticles with a swellable hydrophilic polymer.

The multiparticulate system produced by the above methods can optionally include a controlled release coating. The controlled release coating is added to the multiparticulate system with a fluid bed granulation, for example.

Additional swellable hydrophilic polymer can also be added to multiparticulate system produced by the above methods. The additional swellable hydrophilic polymer can be added to the multiparticulate system with granulation to help blend the components. Granulation can be performed, for example, with a high shear granulator, twin shell blender or double-cone blender, or a simple planetary mixer. The granulated mixture can be screened through a suitably sized mesh screen. A Fitzmill or Co-mill or oscillating mill may be used to control granule size. A V-blender or double cone blender may be used for final blending.

### Methods of Administration

The compositions can be used as pharmaceutical compositions. The compositions can be used for enteral administration, primarily for oral administration. The preparations can be in solid form, for instance, in capsule, powder, or granule, or tablet form.

A composition in the form of a tablet can be prepared using any suitable conventional pharmaceutical additions routinely used for preparing solid compositions. Examples of such additions include, for example, additional carriers, binders, preservatives, lubricants, glidants, disintegrants, flavorants, dyestuffs, and like substances, all of which are known in the art.

A composition in the form of a capsule can be prepared using routine encapsulation procedures, for example, by incorporation of multiparticulate system and excipients into a gelatin capsule.

Any conventional carrier or excipient may be used in the pharmaceutical compositions. The choice of a particular carrier or excipient, or combinations of carriers or excipients, will depend on the mode of administration being used to treat a particular patient or type of medical condition or disease state. In this regard, the preparation of a suitable pharmaceutical composition for a particular mode of administration is well within the scope of those skilled in the pharmaceutical arts. Additionally, the ingredients for such compositions are commercially-available from, for example, Sigma, P.O. Box 14508, St. Louis, Mo. 63178. By way of further illustration, conventional formulation techniques are described in Remington: The Science and Practice of Pharmacy, 20^{th} Edition, Lippincott Williams & White, Baltimore, Md. (2000); and H. C. Ansel et al., Pharmaceutical Dosage Forms and Drug Delivery Systems, 7^{th} Edition, Lippincott Williams & White, Baltimore, Md. (1999).

Representative examples of materials which can serve as pharmaceutically acceptable carriers include, but are not limited to, the following: (1) sugars, such as lactose, glucose and sucrose; (2) starches, such as corn starch and potato starch; (3) cellulose, such as microcrystalline cellulose, and its derivatives, such as sodium carboxymethyl cellulose, ethyl cellulose and cellulose acetate; (4) powdered tragacanth; (5) malt; (6) talc; (7) excipients, such as cocoa butter and suppository waxes; (8) oils, such as peanut oil, cottonseed oil, safflower oil, sesame oil, olive oil, corn oil and soybean oil; (9) glycols, such as propylene glycol; (10) polyols, such as glycerin, sorbitol, mannitol and polyethylene glycol; (11) esters, such as ethyl oleate and ethyl laurate; (12) agar; (13) buffering agents, such as magnesium hydroxide and aluminum hydroxide; (14) pyrogen-free water; (15) isotonic saline; (16) Ringer's solution; (17) ethyl alcohol; (18) phosphate buffer solutions; and (19) other non-toxic compatible substances employed in pharmaceutical compositions.

### Methods of Testing Composition for Release of Active Agent

USP Paddle or Basket Method is the Paddle and Basket Method described, e.g., in U.S. Pharmacopoeia XXII (1990), herein incorporated by reference.

In the methods below, SGF is Simulated Gastric Fluid. SGF can be prepared, as follows. Dissolve 2.0 g of sodium chloride and 3.2 g of purified pepsin that is derived from procine stomach mucosa, with an activity of 800 to 2500 units per mg of protein in 7.0 ml of hydrochloric acid and sufficient water to make 1000 ml. The test solution has a pH of about 1.2.

### Paddle Method

The release of the active agent from the multiparticulate system can be determined by a testing, for example, by the paddle method. In the paddle method, dissolutions runs were performed using USP type 1 or type 2 dissolution test apparatus with a predetermined paddle speed in Simulated Gastric Fluid (SGF), pH1.2 at 37±5° C. At appropriate time interval, samples were withdrawn and analyzed by HPLC.

### Basket Method

The release of the active agent from the multiparticulate system can be determined by a testing, for example, by the basket method. In the basket method, dissolutions runs were performed using a cylindrical basket covered by a mesh. The basket is immersed in Simulated Gastric Fluid (SGF), pH1.2 at 37±5° C., and rotated at a predetermined speed. At appropriate time interval, samples were withdrawn and analyzed by HPLC.

### Representative Profiles

The release profile of the composition can be assessed by the paddle method with simulated gastric fluid (SGF). In certain embodiments, the composition releases about 40% to about 60% of the drug within about 4 hours. In certain embodiments, the composition releases about 70% to about 90% of the drug within about 8 hours. In certain embodiments, the composition releases about 80% to about 95% of the drug within about 12 hours.

### EXAMPLES

The following examples are put forth so as to provide those of ordinary skill in the art with a complete disclosure and description of how to make and use the embodiments, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used.

### Example 1

### Preparation of Baclofen/HPMC Micronized Multiparticulate System

### Materials

Baclofen (Heumann), Methocel K100M CR (Colorcon), Prosolv SMCC, Succinic acid, Avicel 102, EUDRAGIT® NE 30D (bought from Degussa), Celsphere® CP-102, Pharmacoat 606, Syloid® 244 FP, Ethocel 10 FP (Colorcon), Polyvinyl pyrrolidone (PVP) (Sigma Aldrich), Dibutyl Sebacate, Acetone (Fisher Scientific), Isopropyl Alcohol (Lab Safety), Ethyl alcohol (Fisher Scientific)

### Procedure

Baclofen, succinic acid and Prosolv SMCC were blended together in a blender. The blended mixture was passed through a jet mill to obtain particulates with a particle size of about 28 μm. The mixture was blended extra-granularly with Methocel K100M CR, Avicel 102 and Syloid 244 FP. The mixture was than encapsulated in a size 00 capsule. The components of the capsule are shown below.

| Ingredient | % | Mg/capsule |
|---|---|---|
| Baclofen | 39.47 | 180 |
| Prosolv SMCC | | |
| succinic acid | | |
| Pearlitol 200 SD* | 9.21 | 42 |
| HPMC K100M CR (I)* | 16.45 | 75 |
| Syloid 244 FP* | 0.66 | 3 |
| HPMC K100M CR (H)* | 34.21 | 156.01 |
| total | 100 | 456.01 |

*added extragranularly

### Example 2

### Dissolution Profile of HPMC/Baclofen Micronized Multiparticulate System

Dissolutions runs were performed using USP type 2 dissolution test apparatus with paddle speed 100 RPM in Simulated Gastric Fluid (SGF), pH1.2 at 37±5° C. At appropriate time interval, samples were withdrawn and analyzed by HPLC with column Waters Symmetry C18, 4.6×150 mm, UV detection at 265 nm, and the injection volume is 50 μL.

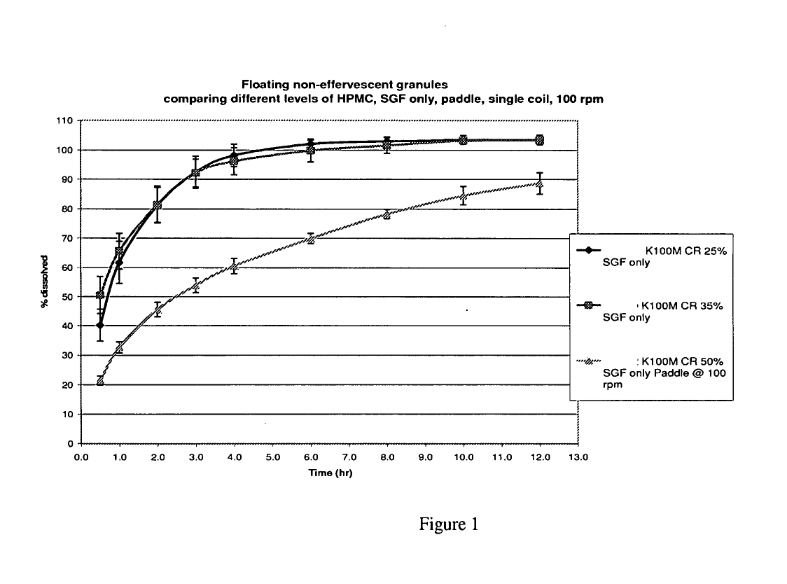

FIG. 1 shows dissolution profiles of a multiparticulate system comprising baclofen and HPMC that was obtained through the mixing/micronization procedure with different amounts of swellable hydrophilic polymer.

In the dissolution runs, the following was observed. As soon as the capsule shell disintegrated and the formulation contacted the dissolution media, the HPMC swelled up and formed a gel-like sticky mass. The particulates started to float within 1 minute of contact with the dissolution medium. Due to the swelling of HPMC, the inner portion of the formulation contained air pockets which gave buoyancy to the composition. Depending on the grade and viscosity of the polymer, the polymer may take a long time to dissolve. Hence the formulation can float for almost 12 hours. It was observed that there was a sustained release property during the in-vitro dissolution run.

### Example 3

### Preparation of Microcrystalline cellulose/Baclofen Coated Multiparticulate System

A coating solution of baclofen, Pharmacoat 606, Syloid 244 FP in a mixture of acetone and isopropyl alcohol was prepared. Microcrystalline cellulose (Celphere CP-102) spheres were coated with the coating solution in a fluid bed granulator. The baclofen-layered spheres were further coated with EUDRAGIT® NE 30 D. The coated spheres were than encapsulated in a size 00 capsule.

The components of the capsule are shown below.

| Ingredient | % | Mg/capsule |
|---|---|---|
| MCC coated with | 60 | 400.6 |
| Baclofen | | |
| EUDRAGIT ® NE 30D | | |
| Talc | | |
| Pearlitol 200 SD* | 14 | 93.5 |
| HPMC K100M CR* | 25 | 166.9 |
| Syloid 244 FP | 1 | 6.7 |
| total | 100 | 667.7 |

*added extragranularly

### Example 4

### Preparation of Ethyl Cellulose/Baclofen Coated Multiparticulate System

A coating solution of baclofen, Pharmacoat 606, Syloid 244 FP in a mixture of acetone and isopropyl alcohol was prepared. A mixture of ethyl cellulose and polyvinyl pyrolidone (PVP) along with dibutyl sebacate as a plasticizer in the form of spheres were coated with the coating solution in a fluid bed granulator.

The coated spheres were blended extra-granularly with Methocel K100M CR, Avicel 102 and Syloid 244 FP. The mixture was than encapsulated in a size 00 capsule.

### Example 5

### Dissolution Profile of Baclofen Coated Multiparticulate System

Dissolutions runs were performed using USP type 2 dissolution test apparatus with paddle speed 100 RPM in Simulated Gastric Fluid (SGF), pH1.2 at 37±5° C. At appropriate time interval, samples were withdrawn and analyzed by HPLC with column Waters Symmetry C18, 4.6×150 mm, UV detection at 265 nm, and the injection volume is 50 μL.

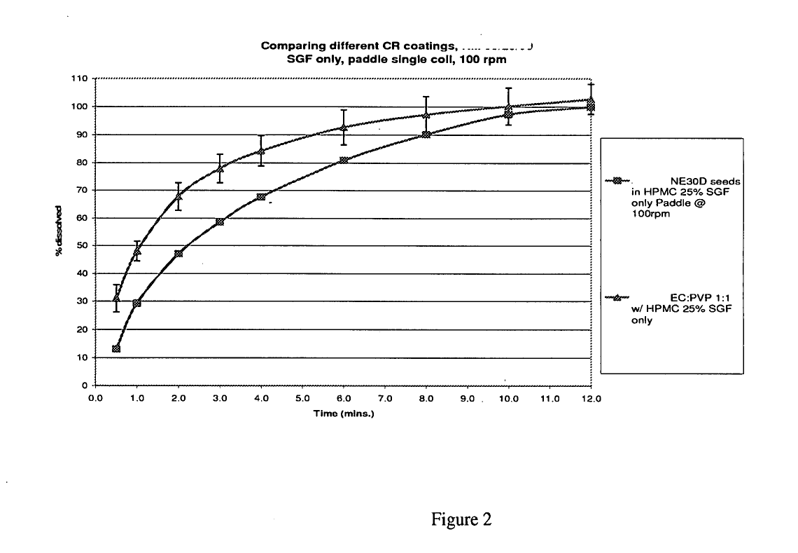

FIG. 2 shows dissolution profiles of a multiparticulate system comprising baclofen multiparticulate system that was obtained through the coated procedure. The compositions tested for FIG. 2 differ by controlled release coatings.

In the dissolution runs, the following was observed. As soon as the capsule shell disintegrated and the formulation contacted the dissolution media, the HPMC swelled up and formed a gel-like sticky mass. The particulates started to float within 1 minute of contacting the dissolution medium. Due to the swelling of HPMC, there was a formation of air pockets which give the formulation the buoyancy. Also, due to the sticky gel mass formed by HPMC due to imbibition of water, the coated seeds containing active agent tended to stick to the formulation and caused it to float with the rest of the mass. The difference between the dissolution profiles of the formulations shown in FIG. 2 is due to the difference in the coating material and the level of coating applied on the baclofen layered seeds. Since the coat for EUDRAGIT® NE 30D is stronger than the ratio of EC:PVP, the dissolution is slower in SGF.

### Example 6

### Comparison of Dissolution Profiles of Micronized and Coated Multiparticulate Systems

Dissolutions runs were performed using USP type 2 dissolution test apparatus with paddle speed 100 RPM in Simulated Gastric Fluid (SGF), pH1.2 at 37±5° C. At appropriate time interval, samples were withdrawn and analyzed by HPLC with column Waters Symmetry C18, 4.6×150 mm, UV detection at 265 nm, and the injection volume is 50 μL.

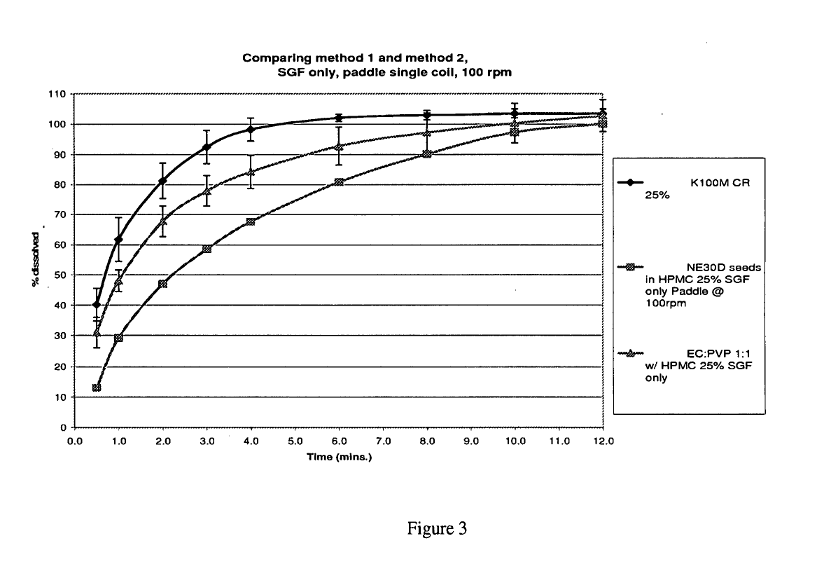

FIG. 3 shows dissolution profiles of a multiparticulate system comprising baclofen multiparticulate system that was obtained through the micronized procedure or coated procedure.

In the dissolution runs, the following was observed. FIG. 3 shows that there is better controlled release with smaller relative standard deviation when the granules are coated rather than including the micronized drug alone in the HPMC blend.

### Example 7

### Comparison of Dissolution Profiles of Micronized and Coated Multiparticulate Systems in SGF and pH 4.5

Dissolutions runs were performed using USP type 2 dissolution test apparatus with paddle speed 100 RPM in Simulated Gastric Fluid (SGF), pH1.2 at 37±5° C. or a solution at pH 4.5. At appropriate time interval, samples were withdrawn and analyzed by HPLC with column Waters Symmetry C18, 4.6×150 mm, UV detection at 265 nm, and the injection volume is 50 μL.

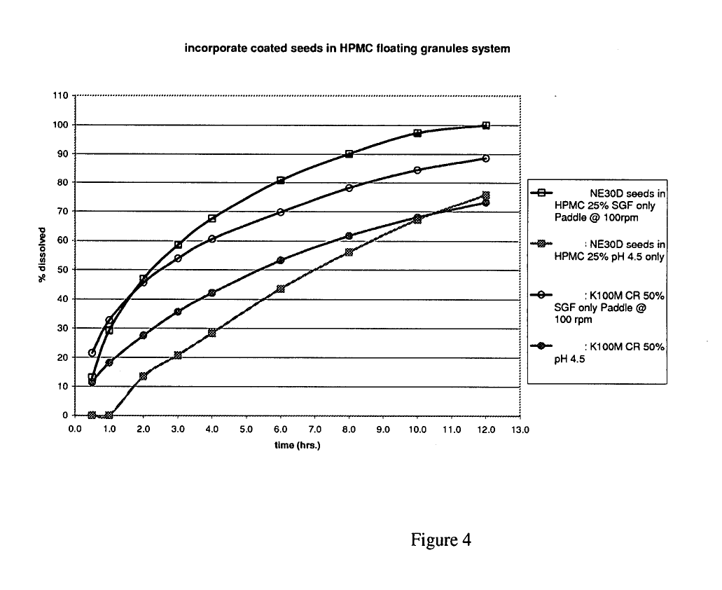

FIG. 4 shows dissolution profiles of a multiparticulate system comprising baclofen multiparticulate system in different dissolution media.

In the dissolution runs, the following was observed. When comparing the dissolution of the formulation in SGF vs pH 4.5, it is observed that the in-vitro release of the drug is slower in pH 4.5. This can be the result of the intrinsic solubility of baclofen decreasing as the pH increases. Since the solubility of the swellable hydrophilic polymer and the controlled release coating materials are independent of pH, the dissolution is controlled by diffusion and erosion of the swellable hydrophilic polymer and is dependant on the solubility of baclofen.

### Example 8

### Comparison of Dissolution Profiles of Micronized and Coated Multiparticulate Systems in Basket or Paddle Methods

In the paddle method, dissolutions runs were performed using USP type 2 dissolution test apparatus with paddle speed 100 RPM in Simulated Gastric Fluid (SGF), pH1.2 at 37±5° C. At appropriate time interval, samples were withdrawn and analyzed by HPLC with column Waters Symmetry C18, 4.6×150 mm, UV detection at 265 nm, and the injection volume is 50 μL.

In the basket method, dissolutions runs were performed using a cylindrical basket covered by a mesh. The basket is immersed in Simulated Gastric Fluid (SGF), pH1.2 at 37±5° C., and rotated at a predetermined speed. At appropriate time interval, samples were withdrawn and analyzed by HPLC.

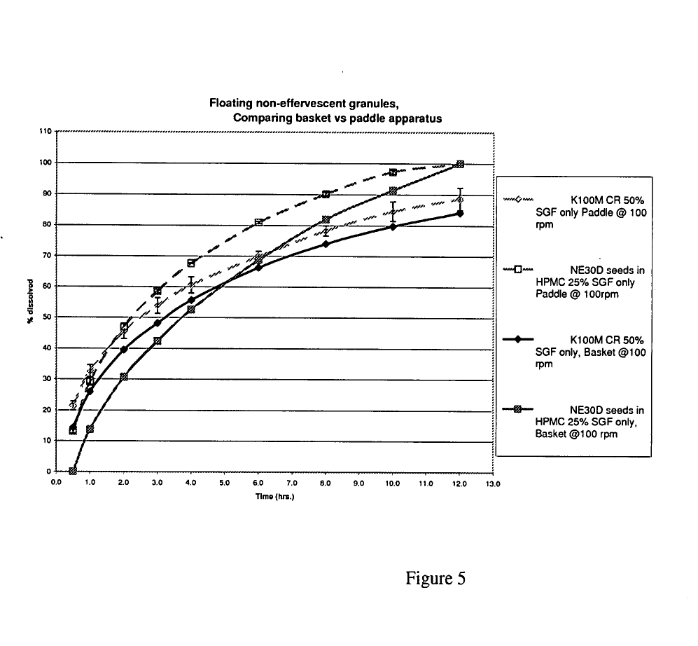

FIG. 5 shows dissolution profiles of a multiparticulate system comprising baclofen multiparticulate system as tested by the basket method and paddle method.

In the dissolution runs, the following was observed. When comparing the in-vitro release in a dissolution apparatus with paddle method compared with basket method, it was observed that due to the nature of the single coil used in the paddle apparatus, the gelled formulation tended to break into a couple of pieces and hence facilitated a comparatively faster dissolution of the formulation. In the basket method, the formulation tended to swell and stick together and hence causing a trapping of the drug for an extended period of time. However, the difference between the paddle method and basket method was not significant.

### Example 9

### Dissolution Profile of HPMC/Levodopa Micronized Multiparticulate System

Dissolutions runs were performed using USP type 2 dissolution test apparatus with paddle speed 100 RPM in Simulated Gastric Fluid (SGF), pH 1.2 at 37±5° C. At appropriate time interval, samples were withdrawn and analyzed by HPLC with column Waters Symmetry C18, 4.6×150 mm, UV detection at 265 nm, and the injection volume is 50 μL.

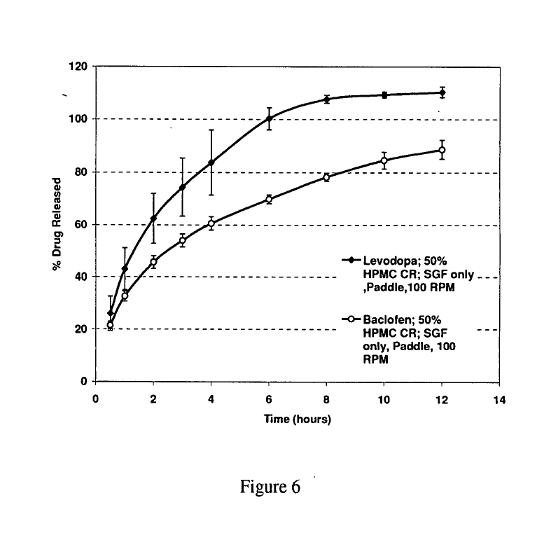

FIG. 6 shows dissolution profiles of a multiparticulate system comprising levodopa and HPMC that was obtained through the mixing/micronization procedure comprising 50% swellable hydrophilic polymer.

In the dissolution runs, the following was observed. As soon as the capsule shell disintegrated and the formulation contacted the dissolution media, the HPMC swelled up and formed a gel-like sticky mass. The particulates started to float within 1 minute of contact with the dissolution medium. Due to the swelling of HPMC, the inner portion of the formulation contained air pockets which gave buoyancy to the composition. Depending on the grade and viscosity of the polymer, the polymer may take a long time to dissolve. Hence the formulation can float for almost 12 hours. It was observed that there was a sustained release property during the in-vitro dissolution run.

### Example 10

### Comparison of Dissolution Profiles of Micronized and Coated Multiparticulate Systems in Basket or Paddle Methods

In the paddle method, dissolution runs were performed using USP type 2 dissolution test apparatus with paddle speed 100 RPM in Simulated Gastric Fluid (SGF), pH1.2 at 37±5° C. At appropriate time intervals, samples were withdrawn and analyzed by HPLC with column Waters Symmetry C18, 4.6×150 mm, UV detection at 265 nm, and the injection volume is 50 μL.

In the basket method, dissolution runs were performed using a cylindrical basket covered by a mesh. The basket is immersed in Simulated Gastric Fluid (SGF), pH1.2 at 37±5° C., and rotated at a predetermined speed. At appropriate time intervals, samples were withdrawn and analyzed by HPLC.

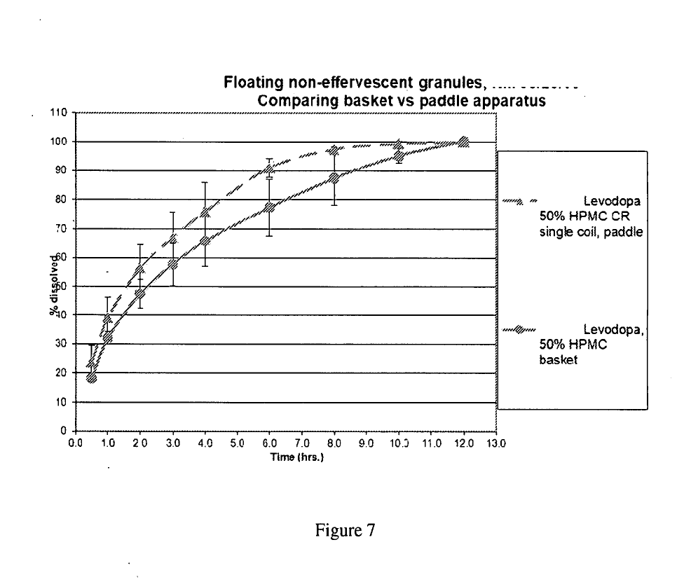

FIG. 7 shows dissolution profiles of a multiparticulate system comprising levodopa as tested by the basket method and paddle method.

In the dissolution runs, the following was observed. When comparing the in-vitro release in a dissolution apparatus with paddle method compared with basket method, it was observed that due to the nature of the single coil used in the paddle apparatus, the gelled formulation tended to break into a couple of pieces and hence facilitated a comparatively faster dissolution of the formulation. In the basket method, the formulation tended to swell and stick together and hence causing a trapping of the drug for an extended period of time. However, the difference between the paddle method and basket method was not significant.

While the present invention has been described with reference to the specific, embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A composition comprising microparticulates comprising a swellable hydrophilic polymer and an active agent, wherein
the swellable hydrophilic polymer is substantially non-crosslinked intramolecularly; and
the size of the microparticulates is about 500 μm or less.

2. The composition of claim 1, wherein the swellable hydrophilic polymer is selected from cellulose polymers and their derivatives, polysaccharides and their derivatives, polyalkylene oxides, polyethylene glycols, chitosan, poly(vinyl alcohol), xanthan gum, maleic anhydride copolymers, poly(vinyl pyrrolidone), starch and starch-based polymers, poly(2-ethyl-2-oxazoline), poly(ethyleneimine), polyurethane hydrogels, and combinations comprising one or more of the foregoing polymers.

3. The composition of claim 1, wherein the swellable hydrophilic polymer is selected from cellulose and derivatives thereof.

4. The composition of claim 1, wherein the swellable hydrophilic polymer is selected from cellulose (such as microcrystalline cellulose), hydroxymethylcellulose, hydroxyethylcellulose (HEC), hydroxypropylmethylcellulose (HPMC), hydroxypropycellulose (HPC), methylcellulose (MC or METHOCEL), ethylcellulose (EC), hydroxyethylmethylcellulose (HEMC), ethylhydroxy-ethylcellulose (EHEC), and carboxymethylcellulose.

5. The composition of claim 1, wherein the swellable hydrophilic polymer is hydroxypropylmethylcellulose (HPMC).

6. The composition of claim 1, wherein the swellable hydrophilic polymer is microcrystalline cellulose or ethylcellulose (EC).

7. The composition of claim 1, wherein the size of the microparticulates is about 300 μm or less.

8. The composition of claim 1, wherein the size of the microparticulates is about 250 μm or less.

9. The composition of claim 1, wherein the size of the microparticulates is about 200 μm or less.

10. The composition of claim 1, wherein the active agent is a Class II, or Class III or Class IV compound, according to the biopharmaceutical classification of drugs in terms of their solubility and intestinal permeability by the FDA.

11. The composition of claim 1, wherein the active agent is baclofen.

12. The composition of claim 1, wherein the active agent is levodopa.

13. The composition of claim 1, further comprising a controlled release coating.

14. The composition of claim 13, the controlled release coating is EUDRAGIT® polymer.

15. A composition comprising microparticulates comprising a swellable hydrophilic polymer and an active agent, wherein
the swellable hydrophilic polymer is substantially non-crosslinked HPMC intramolecularly; and
the size of the microparticulates is about 500 μm or less.

16. A method of preparing a composition comprising microparticulates comprising a swellable hydrophilic polymer and an active agent, wherein the swellable hydrophilic polymer is substantially non-crosslinked intramolecularly; and the size of the microparticulates is about 500 μm or less, the method comprising:
mixing solid swellable hydrophilic polymer and solid active agent.

17. The method of claim 16, further comprising micronizing the solid active agent.

18. A composition produced by the method of any one of claims 16 and 17.

19. A method of preparing a composition comprising microparticulates comprising a swellable hydrophilic polymer and an active agent, wherein the swellable hydrophilic polymer is substantially non-crosslinked intramolecularly; and the size of the microparticulates is about 500 μm or less, the method comprising:
dissolving an active agent in a solution or suspension;
coating a nonpareil seed with the solution or suspension comprising the active agent; and
mixing a solid swellable hydrophilic polymer with the nonpareil seeds coated with active agent.

20. A composition produced by the method of claim 19.

21. A method of preparing a composition comprising microparticulates comprising a swellable hydrophilic polymer and an active agent, wherein the swellable hydrophilic polymer is substantially non-crosslinked intramolecularly; and the size of the microparticulates is about 500 μm or less, the method comprising:
mixing an active agent with a swellable hydrophilic polymer;
wet granulating the mixture of active agent and swellable hydrophilic polymer;
extruding the mixture of active agent and swellable hydrophilic polymer; and
subjecting the mixture of active agent and swellable hydrophilic polymer to spheronization to obtain microparticles.

22. A composition produced by the method of claim 21.

23. A method of preparing a composition comprising microparticulates comprising a swellable hydrophilic polymer and an active agent, wherein the swellable hydrophilic polymer is substantially non-crosslinked intramolecularly; and the size of the microparticulates is about 500 μm or less, the method comprising:
mixing an active agent with an inert polymer;
wet granulating the mixture of active agent and inert polymer;
extruding the mixture of active agent and inert polymer;
subjecting the mixture of active agent and inert polymer to spheronization to obtain microparticles; and
mixing the microparticles with a swellable hydrophilic polymer.

24. A composition produced by the method of claim 22.
